(19) United States
(12) Patent Application Publication
(10) Pub. No.: US 2015/0335684 A1
(43) Pub. Date: Nov. 26, 2015

(54) ORTHOPEDIC USE OF A HYDROGEL COMPOSITION

(71) Applicant: NATIONAL TAIPEI UNIVERSITY OF TECHNOLOGY, Taipei City (TW)

(72) Inventors: Yung-Chin YANG, Taipei City (TW); Shou-Cheng TENG, Taipei City (TW); Wei-Ling HSU, Taipei City (TW)

(73) Assignee: NATIONAL TAIPEI UNIVERSITY OF TECHNOLOGY, Taipei City (TW)

(21) Appl. No.: 14/591,823

(22) Filed: Jan. 7, 2015

(30) Foreign Application Priority Data
May 26, 2014 (TW) 103118233

(51) Int. Cl. A61K 35/33; A61K 47/02; A61K 9/00; A61K 47/36

(52) U.S. Cl. CPC A61K 35/33; A61K 47/36; A61K 47/02; A61K 9/0019

(57) ABSTRACT

A method for augmentation of a soft tissue in a subject, comprising: introducing to the subject a hydrogel composition comprising a porous bio-absorbable ceramic carrier and a hyaluronic acid gel. The porous bio-absorbable ceramic carrier is made of a material selected from the group comprising hydroxyapatite, β-tricalcium phosphate, or calcium polyphosphate. The hydrogel composition further comprises a cell positioned in a pore of the porous bio-absorbable ceramic carrier. The hydrogel composition is introduced into the subject in the form of an injection thereby eliminating the need for a surgical procedure.

### CROSS REFERENCE

This non-provisional application claims priority of Taiwan Patent Application NO. 103118233, filed on May 26, 2014, the content thereof is incorporated by reference herein.

### FIELD OF THE INVENTION

The present invention relates to orthopedic use of a hydrogel composition, and more particularly to use of the hydrogel composition for soft tissue augmentation.

### BACKGROUND OF THE INVENTION

The 21st century is the age of biotechnology, and human medicine requires not only continuous advancements in treatment or prevention of diseases, but also gradual demands on change of appearances. Therefore, orthopedics and dermatology have become an increasingly important field in human medicine.

Soft tissue augmentation is common in orthopedics and dermatology, and divided into autologous transplantation and foreign transplantation by the material used. In autologous transplantation, a natural soft tissue is taken from a part of a body, and then implanted into another part of the same body. The natural soft tissue is readily absorbed by the body so its persistency may be inadequate. In foreign transplantation, a synthetic soft tissue, also called filler, is directly implanted into a part of a body. Since the synthetic soft tissue is not from the body, an immune response can be triggered to reject the synthetic soft tissue. Several materials have been found to be useful for the synthetic soft tissue, for example, a hyaluronic acid gel. The hyaluronic acid gel is too fluid to form a required profile in the body while being implanted into the body. Also, the hyaluronic acid is so bio-absorbable that the hyaluronic acid gel may need to be purposely implanted into the body at an interval of 3-6 months for maintaining the soft tissue augmentation.

Recently, adipose-derived stem cells and hyaluronic acid gels have been combined to be implanted into a body, which is deemed as a combination of autologous transplantation and foreign transplantation. These stem cells can grow to adipocytes in the body; however, the hyaluronic acid gels used herein can't overcome the foregoing problems of difficultly forming a required profile in the implanted body and high bio-absorbability. The hyaluronic acid gels may be further coated on the adipose-derived stem cells, which results in cellular death from hypoxia, or lack of growth factors from lack of contact with the body fluid. The above phenomena lead to inadequate results from the combination technique.

### SUMMARY OF THE INVENTION

The present invention provides a method for augmentation of a soft tissue in a subject. The disclosed method comprises: introducing to the subject a hydrogel composition comprising a porous bio-absorbable ceramic carrier and a hyaluronic acid gel.

### BRIEF DESCRIPTION OF THE DRAWINGS

FIG. 1 is an x-ray diffraction spectrum of β-tricalcium phosphate carriers of Examples 13-16;

FIG. 2A is a scanning electron microscopic picture of a β-tricalcium phosphate carrier of Example 1;

FIG. 2B is a scanning electron microscopic picture of a β-tricalcium phosphate carrier of Example 2;

FIG. 2C is a scanning electron microscopic picture of a β-tricalcium phosphate carrier of Example 3;

FIG. 2D is a scanning electron microscopic picture of a β-tricalcium phosphate carrier of Example 4;

FIG. 3A is a scanning electron microscopic picture of a β-tricalcium phosphate carrier of Example 5;

FIG. 3B is a scanning electron microscopic picture of a β-tricalcium phosphate carrier of Example 6;

FIG. 3C is a scanning electron microscopic picture of a β-tricalcium phosphate carrier of Example 7;

FIG. 3D is a scanning electron microscopic picture of a β-tricalcium phosphate carrier of Example 8;

FIG. 4A is a scanning electron microscopic picture of a β-tricalcium phosphate carrier of Example 9;

FIG. 4B is a scanning electron microscopic picture of a β-tricalcium phosphate carrier of Example 10;

FIG. 4C is a scanning electron microscopic picture of a β-tricalcium phosphate carrier of Example 11;

FIG. 4D is a scanning electron microscopic picture of a β-tricalcium phosphate carrier of Example 12;

FIG. 5A is a scanning electron microscopic picture of a β-tricalcium phosphate carrier of Example 13;

FIG. 5B is a scanning electron microscopic picture of a β-tricalcium phosphate carrier of Example 14;

FIG. 5C is a scanning electron microscopic picture of a β-tricalcium phosphate carrier of Example 15;

FIG. 6 illustrates the porosities of sinters of Examples 1-16;

FIG. 7 illustrates the densities of sinters of Examples 1-16;

FIG. 8 shows the compressive strength of sinters of Examples 1-16;

FIG. 9 shows the viscosity of a hydrogel composition of Example 12;

FIG. 10 presents the activity of various substances against L929 fibroblasts.

All symbols therein are as follows: C, that of a culture medium against the cells; 1, that of a β-tricalcium phosphate carrier of Example 4 against the cells; 2, that of β-tricalcium phosphate carrier of Example 8 against the cells; 3, that of a β-tricalcium phosphate carrier of Example 12 against the cells; 4, that of a β-tricalcium phosphate carrier of Example 16 against the cells;

FIG. 11 presents the cytotoxicity of various substances in L929 fibroblasts. All symbols therein are as follows: C, that of a culture medium in the cells; 1, that of a β-tricalcium phosphate carrier of Example 4 in the cells; 2, that of a β-tricalcium phosphate carrier of Example 8 in the cells; 3, that of a β-tricalcium phosphate carrier of Example 12 in the cells; 4, that of a β-tricalcium phosphate carrier of Example 16 in the cells; LB, that of a lysis buffer in the cells;

FIG. 12A is a scanning electron microscopic picture of L929 fibroblasts on the 1st day after being contacted with a hydrogel composition of Example 12;

FIG. 12B is a scanning electron microscopic picture of L929 fibroblasts on the 3rd day after being contacted with a hydrogel composition of Example 12;

FIG. 12C is a scanning electron microscopic picture of L929 fibroblasts on the 7th day after being contacted with a hydrogel composition of Example 12;

FIG. 13A is a scanning electron microscopic picture of L929 fibroblasts on the 1st day after being contacted with a hydrogel composition of Example 16;

FIG. 13B is a scanning electron microscopic picture of L929 fibroblasts on the 3rd day after being contacted with a hydrogel composition of Example 16; and

FIG. 13C is a scanning electron microscopic picture of L929 fibroblasts on the 7th day after being contacted with a hydrogel composition of Example 16.

### DETAILED DESCRIPTION OF THE INVENTION

Hyaluronic acid, also called hyaluronan or hyaluronate, is a polysaccharide constituted of D-glucuronic acid and N-acetylglucosamine. A hyaluronic acid gel can be of use in eye surgery, for example, corneal transplantation, cataract surgery, glaucoma surgery, or repair for retinal detachment. A hyaluronic acid gel can also be employed as a joint injection or a surgical anti-adhesion. In 2003, U.S. Food and Drug Administration further approved implantation of a hyaluronic acid gel to a body for filling the body's sunken or uneven soft tissue. However, the hyaluronic acid gel is so fluid and bio-absorbable that it is difficult to form a required profile in the body. At least for such reasons, it is advised to implant the hyaluronic acid gel into the body at an interval of 3-6 months.

The present invention provides a method, in which a mixture of porous bio-absorbable ceramic carriers and hyaluronic acid gels exhibits higher viscosity and lower bio-absorbability than the hyaluronic acid gels alone. This mixture can be used in soft tissue augmentation by introducing the mixture to a subject in need thereof, thereby overcoming the problems of high fluidity and bio-absorbability of the hyaluronic acid gels alone. It is noted that the term “soft tissue” used in the content indicates, for example but not limited to, a lip soft tissue, a neck soft tissue, an orbital groove soft tissue, a breast soft tissue, a cheek soft tissue, or a nasal soft tissue.

Therefore, the present invention provides a method for augmentation of a soft tissue in a subject comprising the following step of: introducing to the subject a hydrogel composition comprising a porous bio-absorbable ceramic carrier and a hyaluronic acid gel. A material the carrier is made of is, for example but not limited to, hydroxyapatite, β-tricalcium phosphate, or calcium polyphosphate. Preferably, the carrier has a pore diameter of 5-200 μm. While the hydrogel composition is introduced into the subject, the porous bio-absorbable ceramic carrier can be absorbed by the subject. As such, no adverse response (e.g. immune response) occurs.

The hyaluronic acid gel is mixed with the porous bio-absorbable ceramic carrier. Preferably, the hyaluronic acid gel comprises hyaluronic acid and a physiologically acceptable solvent. An example of the physiologically acceptable solvent is, but not limited to, phosphate buffered saline or water. Since the hyaluronic acid gel partially flows into and/or out of the pore of the carrier, the probability of the hyaluronic acid gel to be in contact with the subject is reduced. That is, the absorbance of the hyaluronic acid gel in the subject is reduced. Further, the hydrogel composition has higher fluidity than the hyaluronic acid gel alone. By such a way, it is convenient to form a required profile in the body. For a better viscosity of the hydrogel composition, the porous bio-absorbable ceramic carrier, the hyaluronic acid, and the physiologically acceptable solvent are preferably in a weight ratio of 1:0.02-0.2:3-19, more preferably in a weight ratio of 1:0.04-0.19:3.96-18.81. Specifically, if the weight ratio is not within the foregoing range, the hydrogel composition may be too viscous or too diluted.

As above, the hydrogel composition of the present invention has higher viscosity and lower bio-absorbability than the hyaluronic acid gels alone, so the hydrogel composition is appropriate to introduce into the subject for soft tissue augmentation. Since the hydrogel composition of the present invention is fluid, it is preferable to be in the form of an injection. Therefore, the hydrogel composition of the present invention can be injected into the subject without any surgical operation on the subject.

The hydrogel composition of the present invention optionally comprises a cell. The cell is positioned in the pore of the carrier, and an example thereof is, but not limited to, a fibroblast, an adipocyte, or an adipose-derived stem cell. According to the properties of the hyaluronic acid gel partially flowing into and/or out of the carrier's pore, the cell will not be covered with the hyaluronic acid gel for a long time, so the cell will not die of hypoxia or lack of growth factors provided by the subject's body fluid.

The following examples are offered to further illustrate the present invention:

### EXAMPLE 1

Production of a β-tricalcium Phosphate Carrier and a Hydrogel Composition

A polymethylmethacrylate microsphere was mixed with a β-tricalcium phosphate powder, and the thus obtained mixture had polymethylmethacrylate of 30 wt % and β-tricalcium phosphate of 70 wt %. After the mixture was well mixed with a zirconium ball and an alcohol solution of 95% in an appropriate amount, the mixture was wetly ground at least for 8 hours. The zirconium ball of the mixture was removed to form a slurry. Then, the slurry was deposited in an oven at 60° C., until the solvent of the slurry was completely removed to obtain a flour. After that, the flour was mixed with polyvinyl alcohol so that the thus obtained blend had the flour of 97 wt % and the polyvinyl alcohol of 3 wt %. The obtained blend was ground and sieved using a 60-mesh sieve to make the blend non-aggregative and its particle size uniform. After which, the blend was deposited in a high-carbon steel mold having a height of 8 cm and a diameter of 0.8 cm, and was compressed into a cylinder. Afterward, the cylinder was heated to 550° C. at a rate of 2° C./min, and stayed at the temperature for 2 hours. The cylinder was further heated to 1,000° C. at a rate of 2° C./min, and stayed at the temperature for 2 hours. Thereafter, the thus obtained sinter was ground and sieved using a 60-mesh sieve to form a β-tricalcium phosphate carrier. Finally, the carrier of 5 g, 10 g, or 20 g was mixed with a hyaluronic acid gel (hyaluronic acid of 1 wt % in water) so that total weight of the thus obtained hydrogel composition is of 100 g. After being heated to 60° C., the hydrogel composition was stirred at this temperature for 2 hours. By this way, the final hydrogel composition was obtained.

### EXAMPLE 2

Production of a β-tricalcium Phosphate Carrier and a Hydrogel Composition

The β-tricalcium phosphate carrier and the hydrogel composition obtained herein were produced according to the procedure described in Example 1, except that the second heating temperature for forming the sinter was of 1,050° C.

### EXAMPLE 3

Production of a β-tricalcium Phosphate Carrier and a Hydrogel Composition

The β-tricalcium phosphate carrier and the hydrogel composition obtained herein were produced according to the procedure described in Example 1, except that the second heating temperature for forming the sinter was of 1,100° C.

### EXAMPLE 4

Production of a β-tricalcium Phosphate Carrier and a Hydrogel Composition

The β-tricalcium phosphate carrier and the hydrogel composition obtained herein were produced according to the procedure described in Example 1, except that the second heating temperature for forming the sinter was of 1,150° C.

### EXAMPLE 5

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 50 wt % and the β-tricalcium phosphate of 50 wt %.

### EXAMPLE 6

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 50 wt % and the β-tricalcium phosphate of 50 wt %, and the second heating temperature for forming the sinter was of 1,050° C.

### EXAMPLE 7

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 50 wt % and the β-tricalcium phosphate of 50 wt %, and the second heating temperature for forming the sinter was of 1,100° C.

### EXAMPLE 8

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 50 wt % and the β-tricalcium phosphate of 50 wt %, and the second heating temperature for forming the sinter was of 1,150° C.

### EXAMPLE 9

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 70 wt % and the β-tricalcium phosphate of 30 wt %.

### EXAMPLE 10

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 70 wt % and the β-tricalcium phosphate of 30 wt %, and the second heating temperature for forming the sinter was of 1,050° C.

### EXAMPLE 11

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 70 wt % and the β-tricalcium phosphate of 30 wt %, and the second heating temperature for forming the sinter was of 1,100° C.

### EXAMPLE 12

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 70 wt % and the β-tricalcium phosphate of 30 wt %, and the second heating temperature for forming the sinter was of 1,150° C.

### EXAMPLE 13

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 90 wt % and the β-tricalcium phosphate of 10 wt %.

### EXAMPLE 14

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 90 wt % and the β-tricalcium phosphate of 10 wt %, and the second heating temperature for forming the sinter was of 1,050° C.

### EXAMPLE 15

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 90 wt % and the β-tricalcium phosphate of 10 wt %, and the second heating temperature for forming the sinter was of 1,100° C.

### EXAMPLE 16

Production of a β-tricalcium Phosphate Carrier and a Hydrogel Composition

The β-tricalcium phosphate carrier and the hydrogel composition obtained herein were produced according to the procedure described in Example 1, except that the mixture had the polymethylmethacrylate of 90 wt % and the β-tricalcium phosphate of 10 wt %, and the second heating temperature for forming the sinter was of 1,150° C.

### EXAMPLE 17

Analysis of x-ray Diffraction

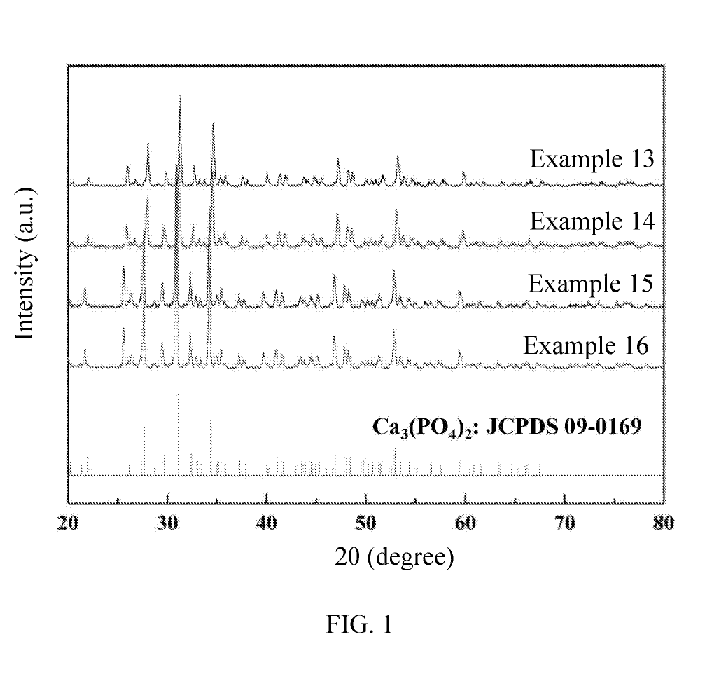

The component of the carriers of Examples 13-16 was determined using an x-ray diffraction meter. The result is shown in FIG. 1. In comparison to JCPDS card #09-0169, the component of the carriers of Examples 13-16 is β-tricalcium phosphate. It is learned that the first heating temperature and the second heating temperature for forming the sinters can't lead to the conversion of their component from β-tricalcium phosphate to α-tricalcium phosphate.

### EXAMPLE 18

### Analysis of Scanning Electron Microscope

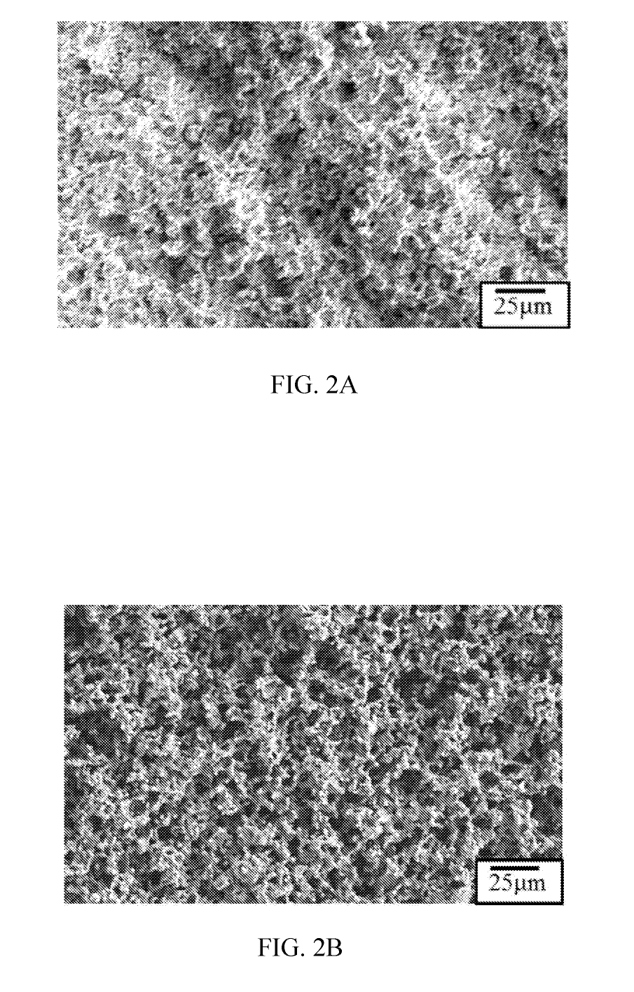

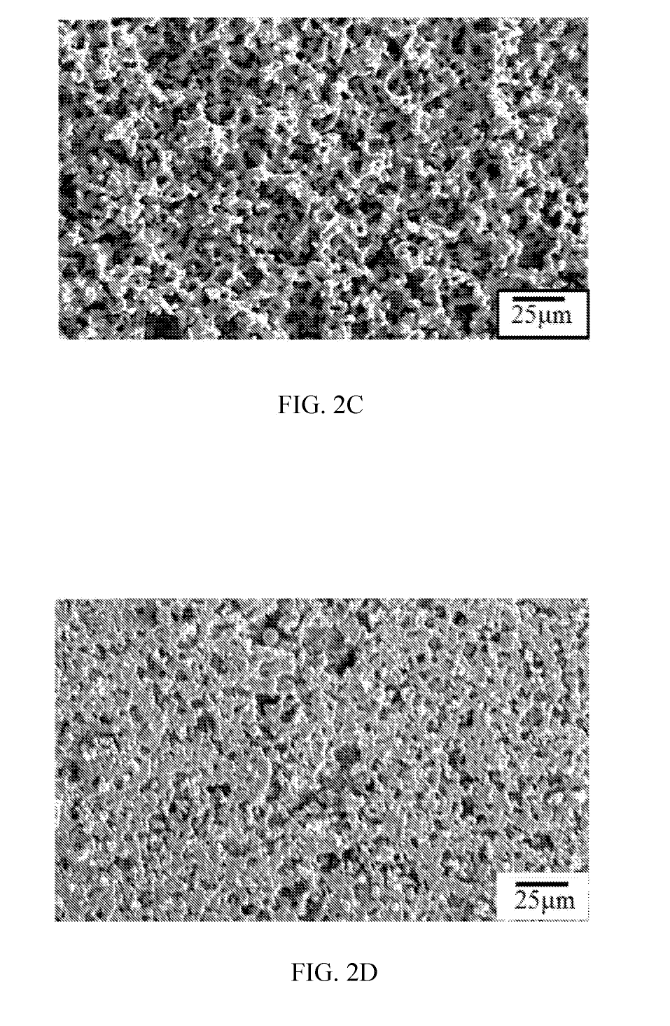

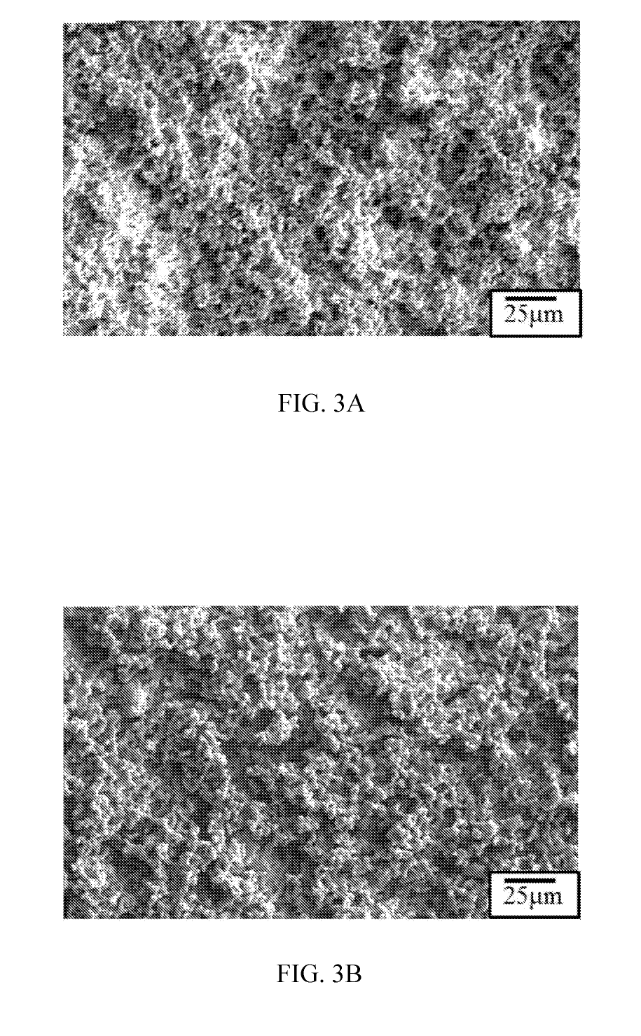

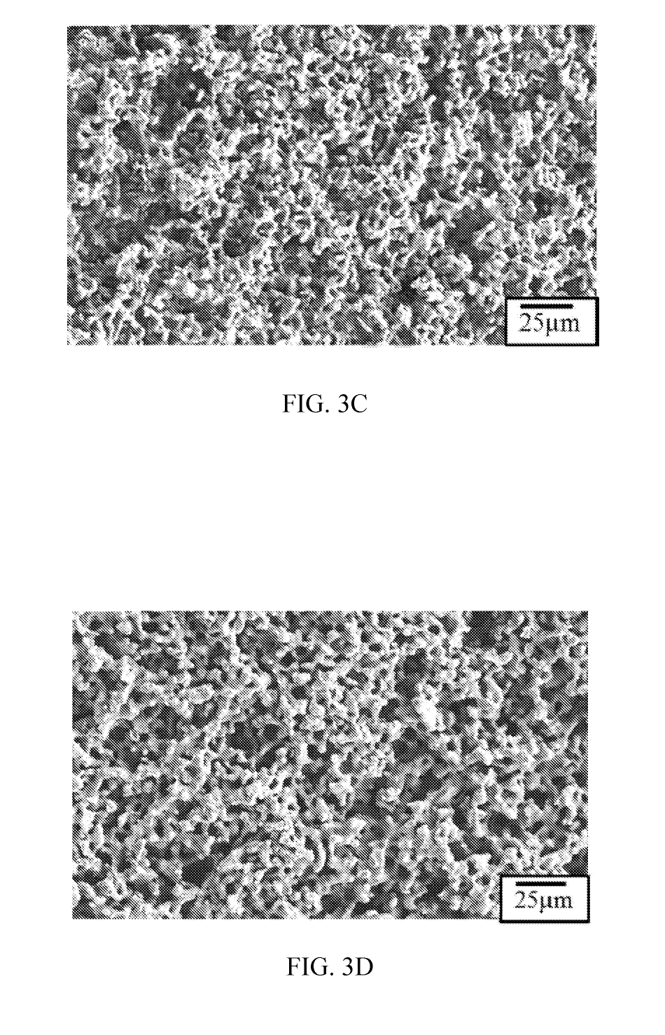

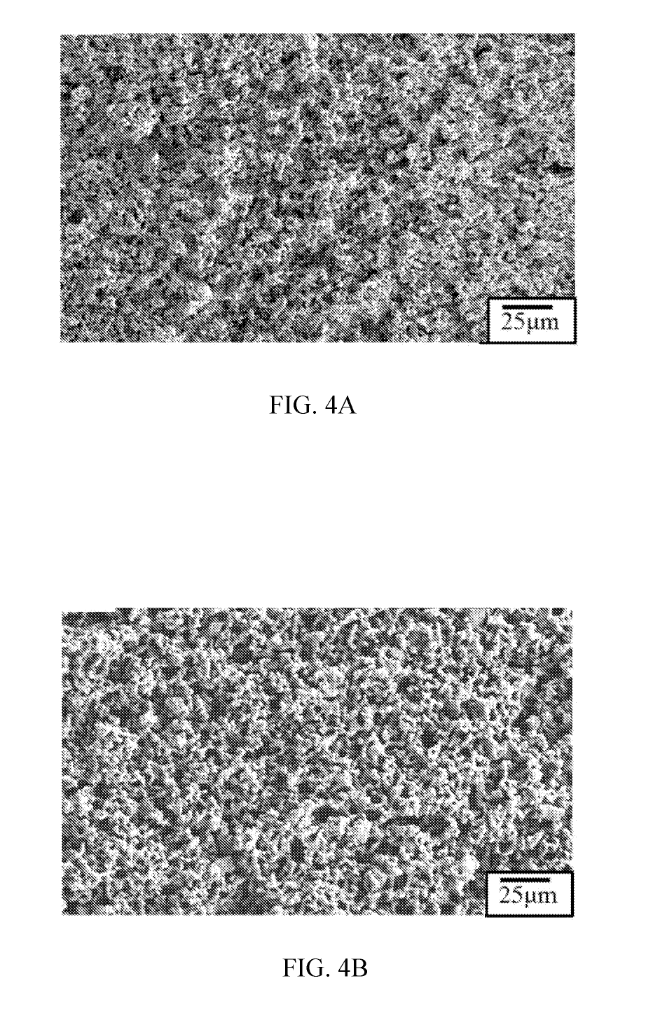

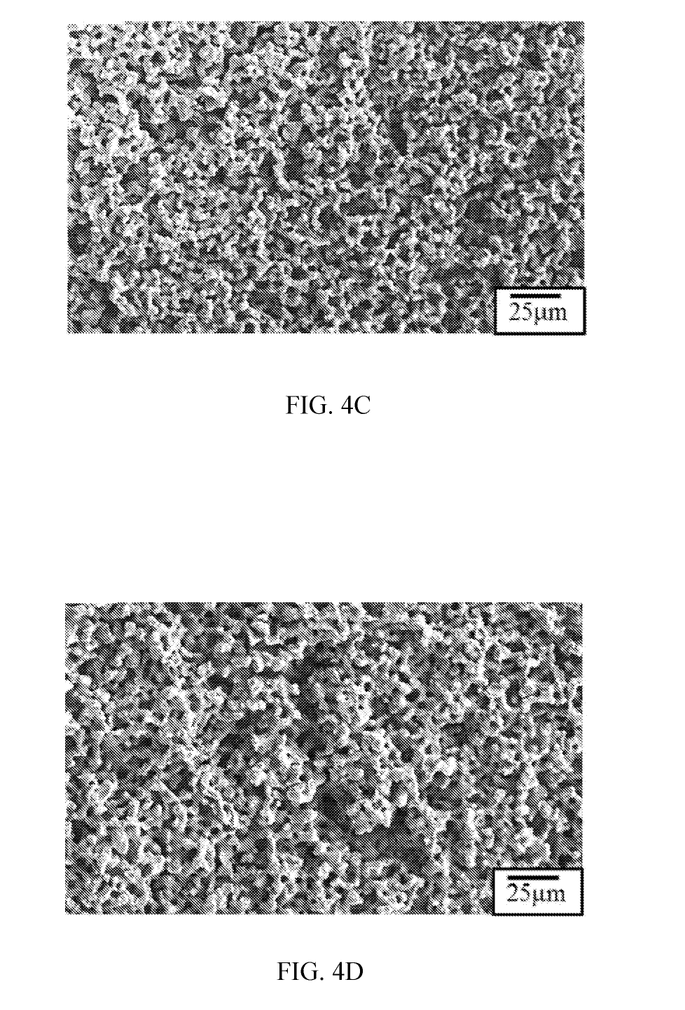

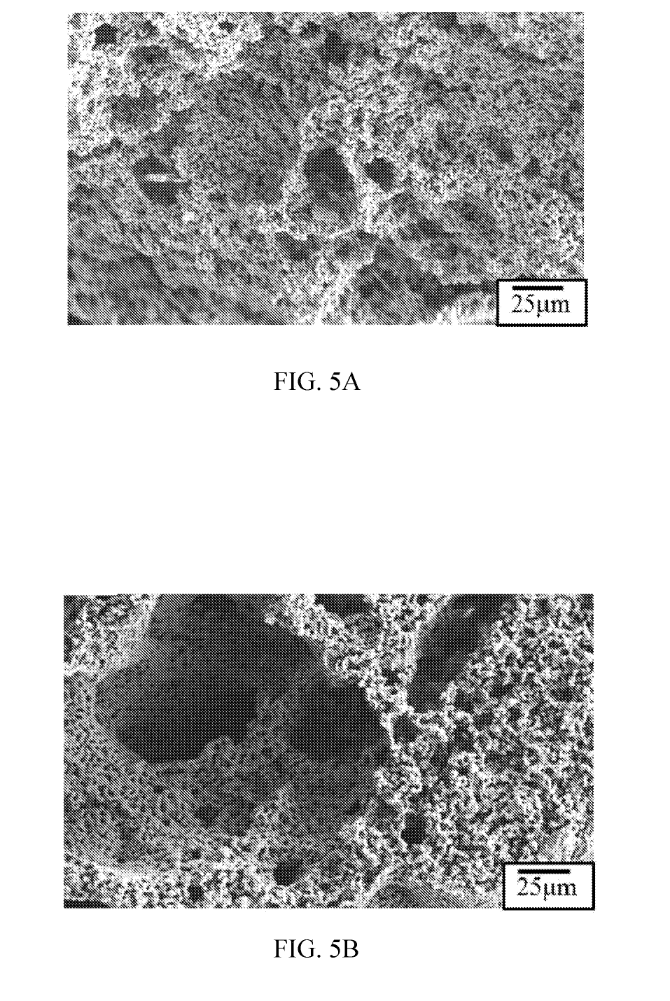

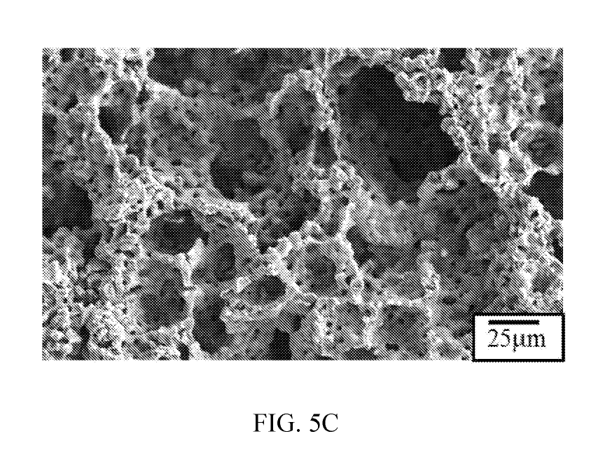

A scanning electron microscope was used to study the morphology of the carriers of Examples 1-15. As shown in FIGS. 2A-2D and 3A-3D, the carriers of Examples 1-8 have similar pore diameters, which indicates that the second heating temperature for forming the sinters has no effect on the pore diameters of the carriers of Examples 1-8, but the compactness of the carriers of Examples 1-8 is improved with the second heating temperature elevated. As shown in FIGS. 4A-4D and 5A-5C, the pore diameters of the carriers of Examples 9-15 are relatively great to those of Examples 1-8. The result suggests that the polymethylmethacrylate concentration and the β-tricalcium phosphate concentration of the mixture may have an effect on the pore diameter of each carrier. As shown again in FIGS. 5A-5C, the pore diameters of the carriers of Examples 13-15 are various. At any rate, it is learned from all of the above that the pore diameters of the carriers of Examples 1-15 range between 5 μm to 200 μm.

### EXAMPLE 19

### Analysis of Archimedes' Method

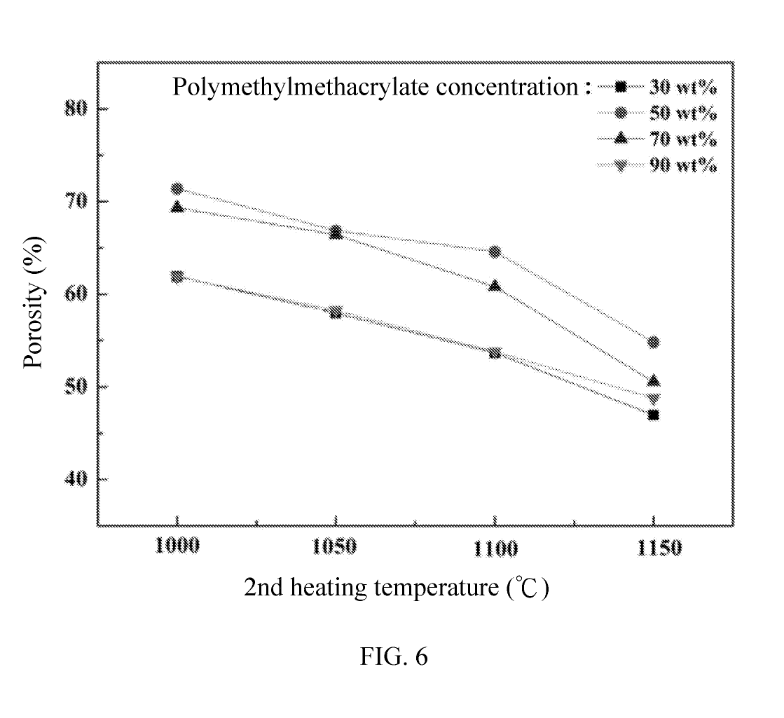

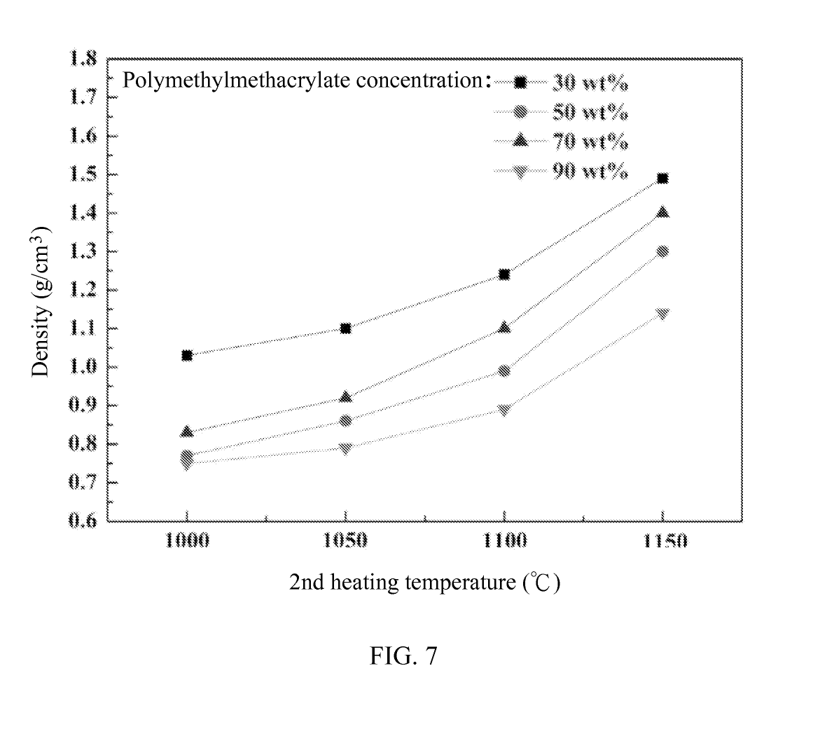

Archimedes' method was introduced to measure the porosities and the densities of the sinters of Examples 1-16. The result of porosities is shown in FIG. 6, and that of densities is shown FIG. 7. With reference to the two FIGS, under the premise that the polymethylmethacrylate concentration and the β-tricalcium phosphate concentration of the mixtures are constant, the porosities of the sinters of Examples 1-16 are reduced and the densities thereof are increased while the second heating temperature is elevated. The above phenomenon is coupled to that the elevated second heating temperature can enhance the compactness of each carrier.

### EXAMPLE 20

### Analysis of Compressive Strength

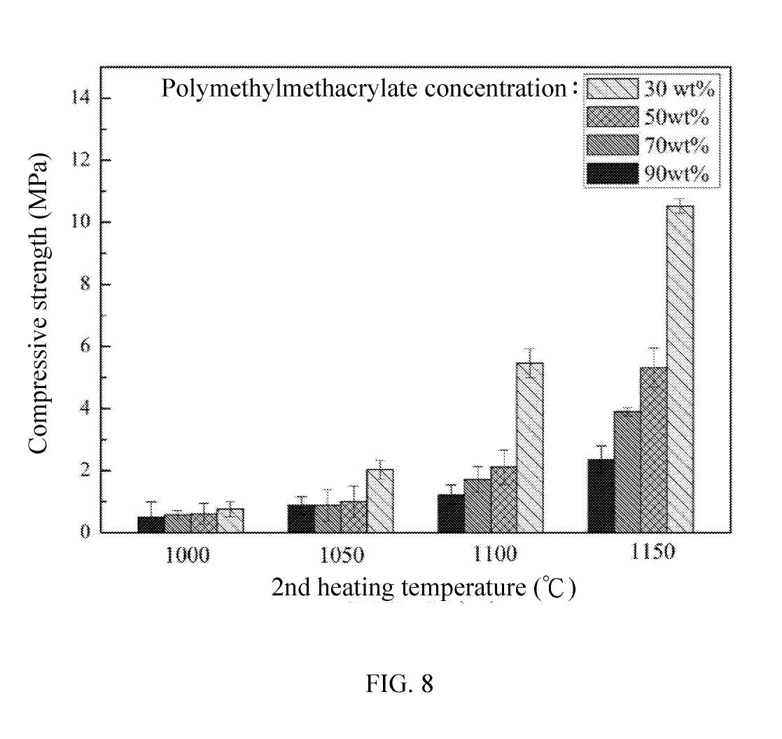

A Shimadzu machine (model NO.: AGS-500D) was used to measure the compressive strength of the sinters of Examples 1-16. As shown in FIG. 8, under the premise that the polymethylmethacrylate concentration and the β-tricalcium phosphate concentration of the mixtures are constant, the second heating temperature can increase the compressive strength of each sinter, which is concert with the increasing of the densities of the sinters by the elevated second heating temperature. Likewise, the above phenomenon is coupled to that the elevated second heating temperature can enhance the compactness of each carrier.

### EXAMPLE 21

### Analysis of Viscosity

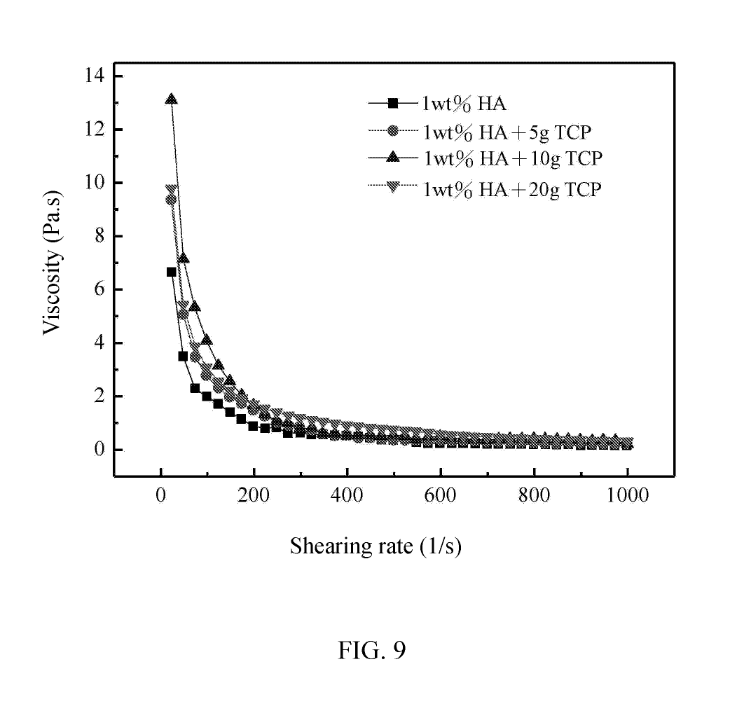

A viscosity testing machine (brand: New Castle, model NO.: AR-1000) was used to analyze the viscosity of the hydrogel composition of Example 12. As shown in FIG. 9, the viscosity of the hydrogel composition of Example 12 is greater than that of a hyaluronic acid gel alone. It is noted that the viscosity is not increased as the weight ratio of the β-tricalcium phosphate carrier to the hyaluronic acid gel, and however, the hydrogel composition containing the β-tricalcium phosphate carrier of 5 wt % exhibits the greatest viscosity.

### EXAMPLE 22

### WST-1 Cell Activity Assay

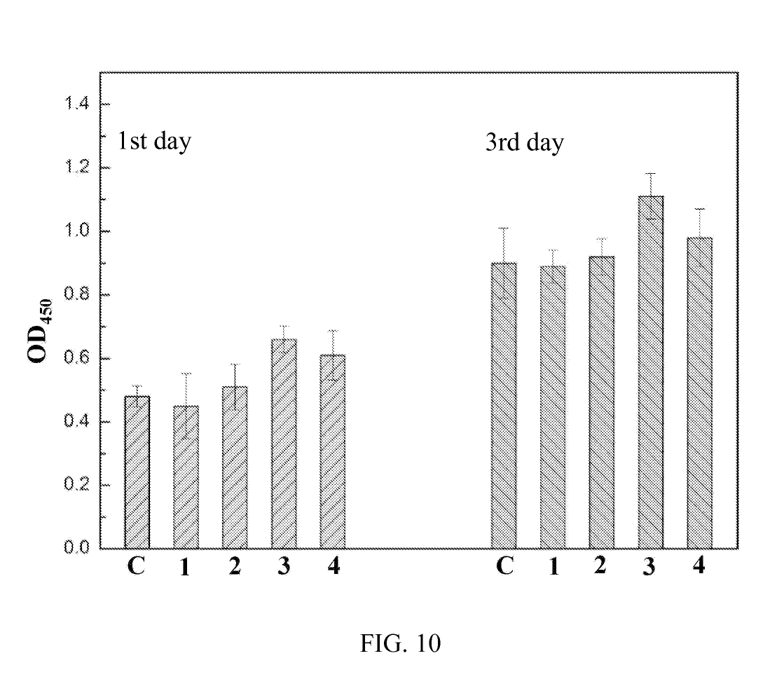

The ISO10993-5 testing method was used to determine the activity of the carriers of Examples 4, 8, 12, and 16 against L929 fibroblasts. As shown in FIG. 10, on the 1st day and 3rd day after each carrier is contacted with the cells, each carrier can't activate the cells. As above, the carriers of Examples 4, 8, 12, and 16 have no bioactivity.

### EXAMPLE 23

### LDH Cytotoxicity Assay

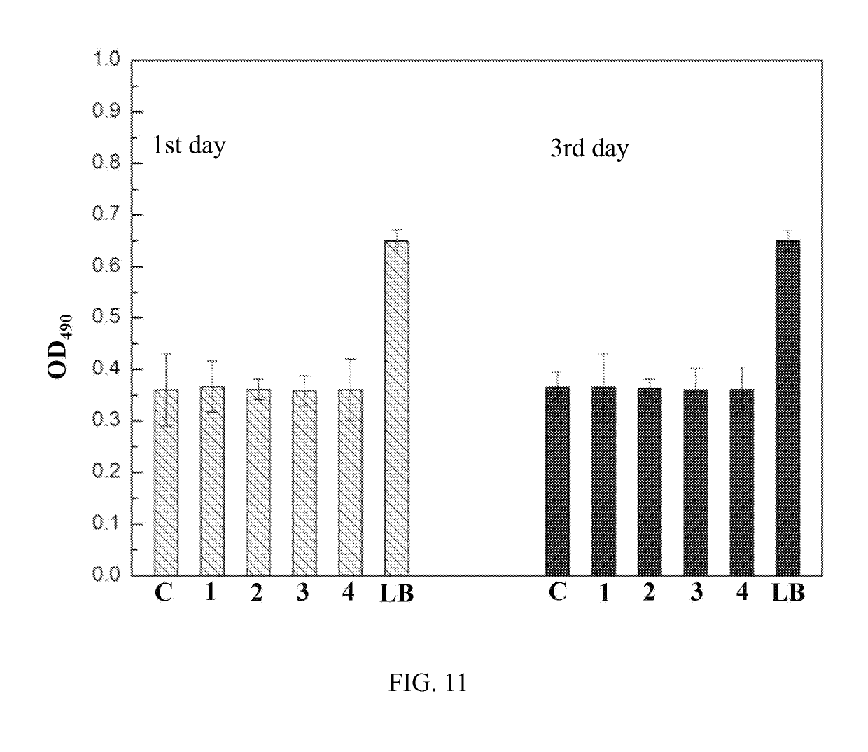

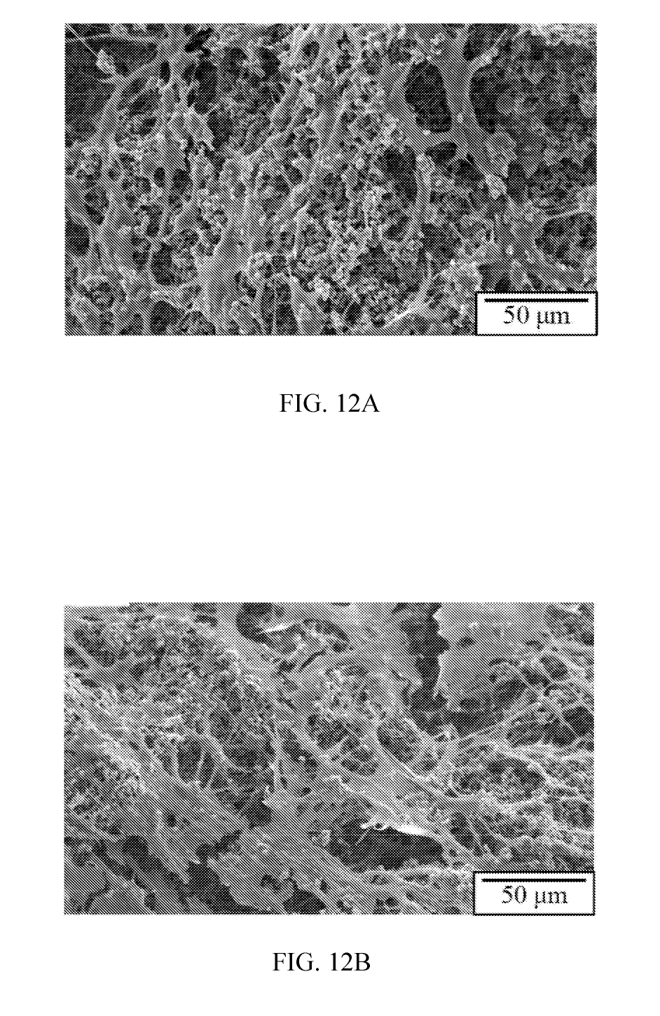

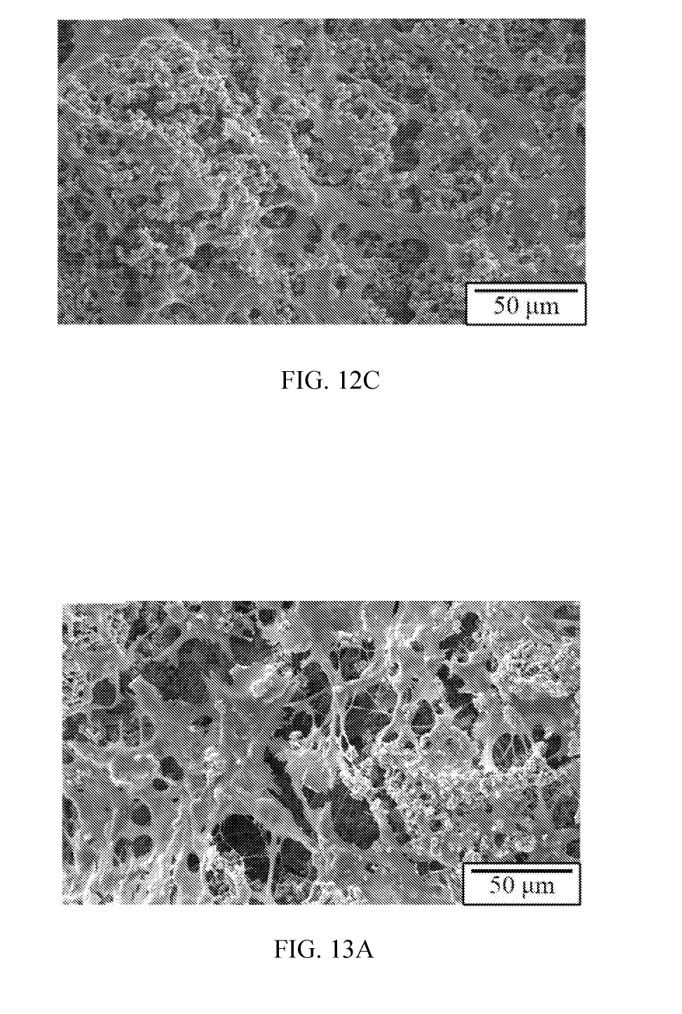

A LDH assay was used to detect the cytotoxicity of the carriers of Examples 4, 8, 12, and 16 in L929 fibroblasts. As shown in FIG. 11, on the 1st day and 3rd day after each carrier is contacted with the cells, the cells are not poisoned with each carrier. As above, the carriers of Examples 4, 8, 12, and 16 are not toxic to the cells.

### EXAMPLE 24

### Cell Adhesion Assay

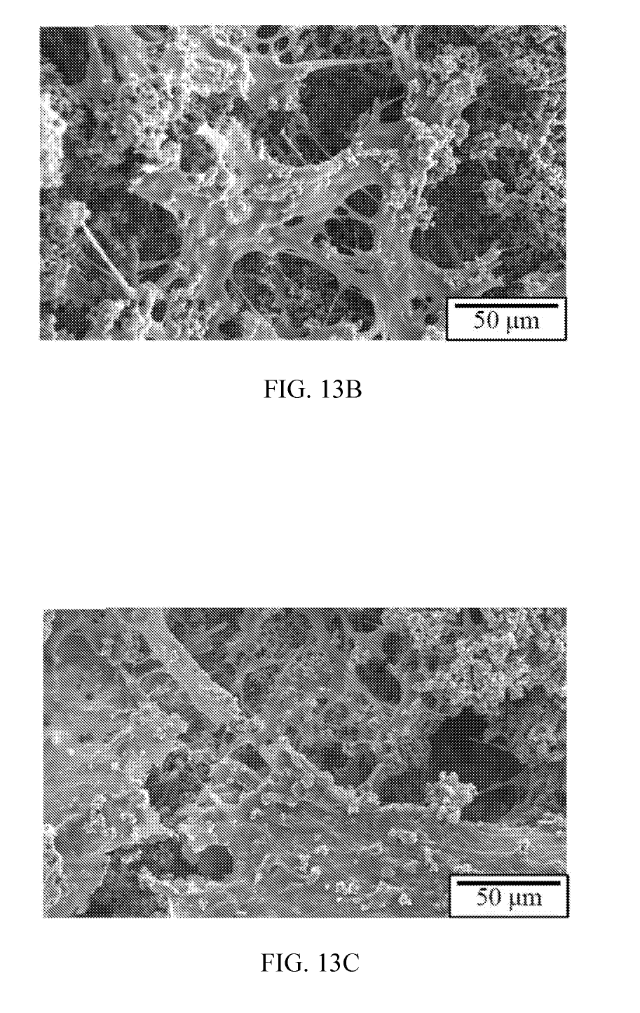

A scanning electron microscope was used to study the contact of the hydrogel compositions of Examples 12 and 16 with L929 fibroblasts. As shown in FIGS. 13A-13C, on the 1st day, 3rd day, and 7th day after the hydrogel composition of Example 12 is contacted with the cells, the cells obviously adhere to the pore of the carrier thereof. Also, the adhesion is more obvious with the contact time increased. As further shown in FIGS. 14A-14C, on the 1st day, 3rd day, and 7th day after the hydrogel composition of Example 16 is contacted with the cells, the cells obviously adhere to the pore of the carrier thereof. Also, the adhesion is more obvious with the contact time increased.

While the invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

1. A method for augmentation of a soft tissue in a subject, comprising:
introducing to the subject a hydrogel composition comprising a porous bio-absorbable ceramic carrier and a hyaluronic acid gel.

2. The method as claimed in claim 1, wherein the porous bio-absorbable ceramic carrier is made of a material selected from the group consisting of hydroxyapatite, β-tricalcium phosphate, or calcium polyphosphate.

3. The method as claimed in claim 1, wherein the porous bio-absorbable ceramic carrier is made of β-tricalcium phosphate.

4. The method as claimed in claim 1, wherein the hydrogel composition further comprises:
a cell positioned in a pore of the porous bio-absorbable ceramic carrier.

5. The method as claimed in claim 2, wherein the hydrogel composition further comprises:
a cell positioned in a pore of the porous bio-absorbable ceramic carrier.

6. The method as claimed in claim 3, wherein the hydrogel composition further comprises:
a cell positioned in a pore of the porous bio-absorbable ceramic carrier.

7. The method as claimed in claim 4, wherein the cell is a fibroblast, an adipocyte, or an adipose-derived stem cell.

8. The method as claimed in claim 5, wherein the cell is a fibroblast, an adipocyte, or an adipose-derived stem cell.

9. The method as claimed in claim 6, wherein the cell is a fibroblast, an adipocyte, or an adipose-derived stem cell.

10. The method as claimed in claim 2, wherein the hyaluronic acid gel comprises:
hyaluronic acid; and
a physiologically acceptable solvent.

11. The method as claimed in claim 3, wherein the hyaluronic acid gel comprises:
hyaluronic acid; and
a physiologically acceptable solvent.

12. The method as claimed in claim 10, wherein the porous bio-absorbable ceramic carrier, the hyaluronic acid, and the physiologically acceptable solvent are in a weight ratio of 1:0.02-0.2:3-19.

13. The method as claimed in claim 10, wherein the porous bio-absorbable ceramic carrier, the hyaluronic acid, and the physiologically acceptable solvent are in a weight ratio of 1:0.04-0.19:3.96-18.81.

14. The method as claimed in claim 11, wherein the porous bio-absorbable ceramic carrier, the hyaluronic acid, and the physiologically acceptable solvent are in a weight ratio of 1:0.02-0.2:3-19.

15. The method as claimed in claim 11, wherein the porous bio-absorbable ceramic carrier, the hyaluronic acid, and the physiologically acceptable solvent are in a weight ratio of 1:0.04-0.19:3.96-18.81.

16. The method as claimed in claim 10, wherein the physiologically acceptable solvent is phosphate buffered saline or water.

17. The method as claimed in claim 11, wherein the physiologically acceptable solvent is phosphate buffered saline or water.

18. The method as claimed in claim 1, wherein the soft tissue is a lip soft tissue, a neck soft tissue, an orbital groove soft tissue, a breast soft tissue, a cheek soft tissue, or a nasal soft tissue.

19. The method as claimed in claim 2, wherein the porous bio-absorbable ceramic carrier has a pore diameter of 5-200 μm.

20. The method as claimed in claim 2, wherein the hydrogel composition is introduced in form of an injection.
